(19) United States
(12) Patent Application Publication
(10) Pub. No.: US 2025/0014730 A1
(43) Pub. Date: Jan. 9, 2025

(54) DIGITAL HUMAN WORKFORCE SOLUTION

(71) Applicant: The Texas A&M University System, College Station, TX (US)

(72) Inventors: Mark Benden, College Station, TX (US); Les Jebson, College Station, TX (US); Kaysey Aguilar, College Station, TX (US)

(73) Assignee: The Texas A&M University System, College Station, TX (US)

(21) Appl. No.: 18/765,535

(22) Filed: Jul. 8, 2024

Related U.S. Application Data
(60) Provisional application No. 63/512,319, filed on Jul. 7, 2023.

(51) Int. Cl. G16H 40/20; G06F 3/0487; G06Q 30/01; G06V 40/16

(52) U.S. Cl. CPC G16H 40/20; G06F 3/0487; G06Q 30/01; G06V 40/174

(57) ABSTRACT

The digital human workplace solution is deployed in a customer service setting and involves a computer programmed with a digital human that is displayed to interact with the customer and adapt to inputs providing information about the user or the environment. The digital human workforce solution is also programmed to interface with one or more databases of information that can be used during the customer service interaction.

### RELATED APPLICATION

The present application claims the benefit of U.S. Provisional Application No. 63/512,319, filed Jul. 7, 2023, which is herein incorporated by reference in its entirety.

### FIELD OF THE INVENTION

The invention relates generally to providing digital representations of service providers in a computing environment that react to inputs received from the user or the environment.

### BACKGROUND

There currently are acute human workforce shortages for positions that require interactions with individuals. These types of customer service positions experience high turnover due to factors such as low pay, time pressures, and negative interactions with individuals. For example, clinical practices typically see a high turnover in receptionist positions. Other examples include positions that require the intake of information from individuals such as workers at the border trying to process immigrants or positions that are designed to directly provide information to individuals such as a campus director trying to help students navigate a campus during orientation.

These customer service positions are typically one of the first interactions of the individual with the business, clinic, governmental unit, or university and if that experience is negative for the individual, can limit the information, or the truthfulness or accuracy of the information, exchanged by the individual throughout his or her interactions with the business, clinic, governmental unit, or university.

Current solutions for such workforce shortages involve hiring temporary workers or using computers to intake information. Temporary workers often lack the training necessary to respond to the emotional state of the individual from whom they are trying to collect information. Computers that simply intake information are also not responsive to the individual from whom the information is being collected. The lack of responsiveness, and inability to adapt to inputs from the user or the environment, creates a robotic, impersonal collection of information rather than a dynamic exchange of information particularly relevant to the individual.

The present invention solves these problems by creating a digital human workforce solution that adapts to inputs providing information about the user or the environment to respond to and collect information from individuals.

### SUMMARY OF THE INVENTION

The digital human workforce solution is deployed in a customer service setting and involves a computer programmed with a digital human that is displayed to interact with the customer and adapt to inputs providing information about the user or the environment. The digital human workforce solution is also programmed to interface with one or more databases of information that may be used during the customer service interaction.

For example, the digital workforce solution may be deployed in a medical practice or dental practice setting. The solution allows the clinical practice to continue to effectively operate by receiving and interacting with patients for their appointments, and allows for collection of outstanding historical or current expenses associated with their care. This may be achieved by utilizing a kiosk or other comparable peripheral in a clinical practice with a fully AI programmed digital human that is interfaced with an electronic health record system. The digital human serves as a customer service representative that is able to schedule new or follow up patient appointments, accept outstanding balances, process co-pays and validate patient demographics and insurance details automatically. The digital human does so in an empathetic way that provides a similar experience to a human customer service representative without bias.

In one embodiment, the digital human workplace solution system comprises a computer containing program instructions on a computer-readable medium for displaying a digital human that is programmed to interact with a user, a display, and one or more devices to provide input data, such as, but not limited to, a keyboard, a mouse, a pulse oximeter, a thermometer, a camera, a microphone, or a floor scale. Input devices may be connected to the digital human workplace solution system by either wired or wireless connection. The display may be a touchscreen programmed to accept user input by touch or may be a screen that does not accept touch input.

If the digital human workplace solution contains a mouse, the mouse may be equipped with one or more of the following sensors: an accelerometer, a skin-temperature sensor, a pulse oximeter, or one or more galvanic-skin resistance sensors. An accelerometer may be used to collect data regarding the user's motions. A skin-temperature sensor may be used to collect user temperature data. A pulse oximeter sensor may be used to collect data used by a computer to determine the oxygen saturation level of the user. One or more galvanic-skin resistance sensors may be used to collect data used by a computer to measure the galvanic skin response. The computer may be further programmed to use the galvanic skin response data to detect whether the patient may be experiencing stress, such as anxiety.

In one embodiment, the digital human workplace solution comprises a computer containing program instructions on a computer-readable medium for displaying a digital human that is programmed to interact with a user, means for receiving data from input devices, and a database to store information.

In one embodiment, the digital human workplace solution comprises a computer containing program instructions on a computer-readable medium for displaying a digital human that is programmed to interact with a user, a display, and a thermometer, such as a thermal scan, wherein the computer receives temperature data from the thermometer and determines whether the user's temperature is above a certain threshold, such as 98.6 degrees Fahrenheit or 100.4 degrees Fahrenheit. The computer is further programmed such that if the user's temperature is above the threshold, the digital human asks the user questions seeking additional input regarding symptoms or causes of the fever.

In one embodiment, the digital human workplace solution comprises a computer containing program instructions on a computer-readable medium for displaying a digital human that is programmed to interact with a user, a display, and a pulse oximeter, wherein the computer is programmed to receive data from the pulse oximeter and automatically record the oxygen saturation of the user in an electronic medical record.

In one embodiment, the digital human workplace solution comprises a computer containing program instructions on a computer-readable medium for displaying a digital human that is programmed to interact with a user, a display, and a floor scale, wherein the computer is programmed to receive data from the floor scale and automatically record the weight of the user in an electronic medical record.

In one embodiment, the digital human workplace solution comprises a computer containing program instructions on a computer-readable medium for displaying a digital human that is programmed to interact with a user, a display, and a camera, wherein the computer is programmed to receive data from the camera, process that data to determine at least one facial expression of the user, and automatically cause the displayed facial expression of the digital human to match the determined facial expression of the user. For example, if the patient is smiling, the computer will automatically modify the displayed facial expression of the digital human to smile. Alternatively, if the patient is furrowing their brow in concern, the computer will automatically modify the displayed facial expression of the digital human to furrow its brow in concern.

In one embodiment, the digital human workplace solution comprises a computer containing program instructions on a computer-readable medium for displaying a digital human that is programmed to interact with a user, a display, and a keyboard. Optionally, the keyboard may include a wrist support that includes one or more sensors to measure conductivity.

In one embodiment, the digital human workplace solution comprises a computer containing program instructions on a computer-readable medium for displaying a digital human that is programmed to interact with a user, a display, and a photoplethysmogram, wherein the computer is programmed to receive data from the photoplethysmogram, determine the respiration rate of the user, and automatically record the respiration rate of the user in an electronic medical record.

The invention also includes a method for programming a digital human system involving the steps of: (i) programming the external appearance of a digital human; (ii) programming the personality of the digital human; (iii) programming the dialog flow for user interaction(s); and (iv) programming how the digital human will respond to data received from one or more devices.

In one embodiment, the digital human workplace solution system comprises a computer containing program instructions on a computer-readable medium for displaying a digital human that is programmed to interact with a user, a display, one or more devices to provide input data, and program instructions on a computer-readable medium for one or more of: natural language processing, converting speech to text, or converting text to speech.

In one embodiment, the digital human workplace solution system comprises a computer containing program instructions on a computer-readable medium for displaying a digital human that is programmed to interact with a user, a display, one or more devices to provide input data, and one or more devices for payment collection. A device for payment collection may include both software and hardware for a point-of-sale transaction.

The invention further comprises a computer-implemented method comprising: (i) sending a digital human for display; (ii) receiving data about a user from one or more input devices; (iii) adjusting the dialog of the digital human in response to the received data; and/or (iv) adjusting the facial expressions of the digital human to mirror the facial expressions of the user.

The invention further comprises a computer-implemented method comprising: (i) programming the external appearance of a digital human; (ii) programming the personality of the digital human; (iii) programming the dialog flow for one or more user interactions; and (iv) programming how the digital human will response to data received from one or more devices. Optionally, the step of programming the dialog flow may include programming the computer to use artificial intelligence to determine the dialog flow in response to user input.

The invention further comprises a computer program product comprising instructions, which when the program is executed by a computer, causes the computer to carry out the method of: (i) sending a digital human for display; (ii) receiving data about a user from one or more input devices; (iii) adjusting the dialog of the digital human in response to the received data; and/or (iv) adjusting the facial expressions of the digital human to mirror the facial expressions of the user.

### BRIEF DESCRIPTION OF THE DRAWINGS

FIG. 1 depicts an exemplary display of a digital human according to aspects of the disclosure.

FIG. 2 depicts a block diagram of an exemplary digital human kiosk for use in a clinical setting according to aspects of the disclosure.

FIG. 3 depicts considerations for decisions regarding programming a digital human as a workplace trainer.

### DETAILED DESCRIPTION OF THE INVENTION

Various embodiments will now be described more fully with reference to the accompanying drawings. The disclosure may, however, be embodied in many different forms and should not be construed as limited to the embodiments set forth herein. In addition, one of skill in the art could modify one or more embodiments disclosed herein to include features described in one or more other embodiments based on the disclosure herein.

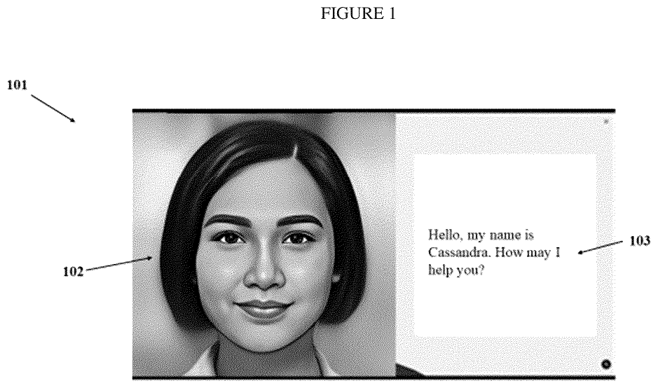

“Digital human” as used herein refers to the digital avatar of a human, which is programmed to interact with a user. For example, FIG. 1 depicts a digital human 102. Conversation-based interactions between a digital human and a user may occur through the use of speech-to-text and text-to-speech technology.

“Digital human workplace solution” refers to the system that implements the software of the digital human, including at a minimum, a computer containing program instructions on a computer-readable medium, a display, software for the digital human, and one or more inputs to collect information from or about a user.

FIG. 1 depicts an exemplary display 101 showing digital human 102. The digital human 102 is programmed to interact with a user by asking questions, either verbally through a speaker or visually on the display through text, preferably, the digital human 102 communicates with the user both verbally and visually. Digital human interactions with a user may involve input from a user that triggers an output from the digital human, but instead of words on a screen, a digital human may respond to spoken input with audio output. Further, the digital human may be programed to provide empathetic interactions through its ability to display emotion and react to facial expressions. For example, the digital human may be programmed to alter facial features in response to negative subject matter presented during an interaction with the user. Further, the digital human 102 may be programmed to display a general personality type such as bubbly, conscientious, shy, or elated.

The digital human 102 may be programmed as an outward facing digital customer service representative that appears three-dimensional and has facial movements that correspond to the verbal communication it provides to the user. An exemplary shell for the digital human 102 may be provided by, for example, Soul Machines Inc. or SapientX, and artificial intelligence used to power how the digital human 102 interacts with the user may be provided by IBM Watson. Examples of types of customer service representatives that the digital human 102 may be programmed as, include, but are not limited to, virtual assistants, coaches, hotel clerks, administrative assistants, product consultants, brand ambassadors, retail assistants, or workplace trainers.

The digital human 102 may also be programmed, through the digital human platform provider, to assume different facial appearances, voices, and personality styles. Within the Natural Language Programming (“NLP”), its speech can be programmed to display specific facial features and gestures during words or sentences. The appearance of the digital human 102 may be customized. For example, hair, eye, and skin color may be programmed, as well as the size and style of different facial features (i.e., eye, lip, face, eyebrow, and nose shape).

Through application program interfaces, the digital human 102 may be integrated with external databases or websites, displaying or altering information from external sources. Additionally, other design decisions-like the digital human's name or their personalized script-must be made in accordance with the purpose of the use-case. The unique integration of these options is informed by task analysis, usability testing, research with digital humans, and experience with client and patient interactions. This developer knowledge informs platform integration and design choices that create a digital human experience which meets the purpose of the interaction, characteristics of the user base, and brand of the organization.

In addition, the digital human 102 may be programmed to use different languages depending on the user. For example, if spoken Spanish is detected in the immediate proximity of the digital human workplace solution, the digital human 102 may ask the user whether they would prefer to speak in Spanish. As another example, the digital human 102 could be programmed to speak in sign language for a user known or detected to have a hearing impairment. The digital human 102 may be programmed to communicate in multiple languages via Natural Language Programming code available as open source that would be coded for each unique application, conversation and Application Programing Interface (“API”) connections to services such as financial, clinical, or personal. Examples of languages that the digital human may be programmed to use include: Spanish, French, German, Italian, English (including British, American, or Australian), Swedish, Norwegian, Danish, Lithuanian, Latvian, Mandarin Chinese, Cantonese, Vietnamese, Hindi, Arabic, Bengali, Tamil, Russian, Portuguese, Indonesian, Urdu, Japanese, Punjabi, Javanese, Wu Chinese, Telugu, Turkish, Korean, Marathi, Swahili, Amharic, Yoruba, Oromo, Hausa, Igbo, Zulu, Shona, or sign language, including American Sign Language.

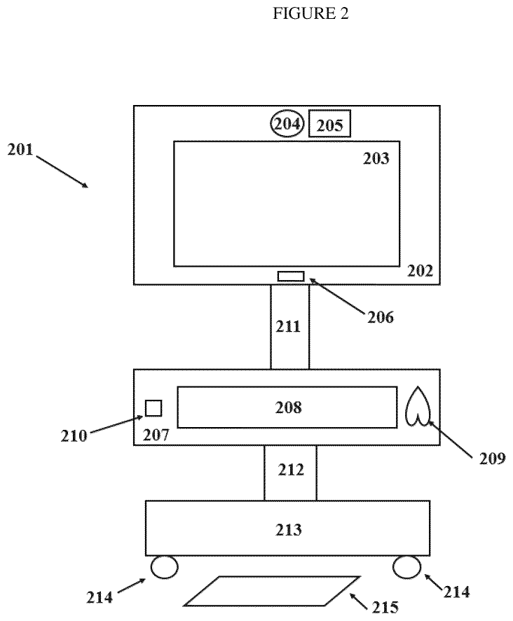

FIG. 2 depicts a block diagram of an exemplary digital human workplace solution as a kiosk 201 for use in a clinical setting. The clinical setting may be a medical office, a dental office, an emergency room, or the like. In this embodiment, a computer 202 is programmed to control one or more devices connected, either through wired or wireless connections to the computer 202, including a display 203, a camera 204, an infra-red thermometer 205 (containing a red laser and detection sensor, not depicted), a microphone 206, a keyboard 208, a mouse 209, a pulse oximeter 210, and a floor scale 215.

The computer 202 is programmed to display the digital human and may communicate with one or more server computers to access information from one or more databases. For example, the digital human workplace solution may be programmed to communicate with one or more server computers that include electronic health record (EHR) or electronic medical record (EMR) software, which helps healthcare providers manage patient medical records and automate clinical workflows, in order to update patient medical records based on the measurements taken while the user is interacting with the digital human. For purposes of this disclosure, “medical record,” “electronic health record,” “electronic medical record,” and “patient medical record” are used interchangeably.

Due to privacy concerns and regulations, data, software, and/or programming related to electronic medical records and one or more digital humans, including large language models used with artificial intelligence to power the interaction of a digital human with a user, may be maintained behind a firewall at each physical location on which a digital human workplace solution is present. Alternatively, data, software, and/or programming relating to electronic medical records and one or more digital humans may be accessible through the use of cloud computing.

The digital human workplace solution includes a display 203 on which the digital human may be presented to the user. In FIG. 2, display 203 is depicted as a computer monitor. The display of the digital human workplace solution could be any screen capable of displaying the digital human, for example, the display could also be a television or tablet. The display may also be a touchscreen from which the computer 202 may receive user inputs. Further, the computer 202 may be programmed to adapt the size of the text used on the display to the needs of the individual user. For example, if the user is known to have an eyesight impairment, the font size of the text may be increased significantly.

When used with certain digital humans, the digital human workplace solution may also be equipped with a camera 204. The camera 204 may provide feedback about the user to the digital human, which can be programmed to use that feedback (along with feedback from the microphone 206) to interpret sentiment, facial expressions, and emotion to change tone, speed of talk, or volume accordingly. The digital human 102 may also be programmed to respond to emotions of the user such as, interest, joy, surprise, sadness, anger, disgust, contempt, self-hostility, fear, shame, shyness, and guilt. Consequently, if a user's facial expressions appear positive, neutral, or negative, the digital human's facial expressions will automatically adjust to mirror the user's facial expressions. Or, specifically for negative speech, if a user's speech content is negative, the digital human can adjust its facial expression to appear empathetic.

The digital human workplace solution may also be equipped with an infra-red thermometer 205, which is used to take measurements to calculate the user's temperature. Depending on whether the user is running a fever, the digital human may ask different questions of the user to try and identify potential causes of the fever. In addition, in non-clinical settings, such as at the border, the temperature measurement may be used to screen users who may be ill and flag those individuals for specific follow-up.

The digital human workplace solution may also be equipped with a microphone 206. The microphone 206 may be used with speech-to-text software in order to translate what the user is saying to the digital human into a form that can be used by the computer 202 to record information or cause the digital human to change the tone of interaction with the user. The microphone 206 may also be used with software programmed to recognize slurring of speech. If slurring of speech is recognized, the digital human may ask the user for additional information used to determine whether the user is drunk, experiencing a blood sugar level at a high or low extreme, or having a stroke.

Ideally, the digital human workplace solution includes a natural language processing system (“NLP”) to process spoken or written language and a system for speech-to-text (“STT”) and text-to-speech (“TTS”) to convert inputs and outputs during interactions between the digital human and one or more users. Examples of those systems include, for example, Google Dialogflow, Google STT, and Google TTS, respectively. Alternatively, other artificial intelligence services may be used such as IBM Watson, Amazon Lex, or Microsoft Azure to assist with programming the digital human.

If the display is a touchscreen, the user may be able to provide some inputs through the display. The digital human workplace solution may also be equipped with a digital keyboard 208 and mouse 209, which allow the user to input information or respond to questions.

The keyboard 208 and mouse 209 may optionally be each equipped with one or more sensors that is electrically coupled to the computer to measure performance or biometric parameters according to PCT/US202/049865, which is herein incorporated by reference in its entirety. The digital human may request that the user use the mouse and keyboard in a specific way to obtain baseline measurements from the sensors or to compare the measurements with a previous baseline set of measurements. For example, the mouse or keyboard may be equipped with one or more of the following sensors: an accelerometer, a skin-temperature sensor, a pulse oximeter, or a galvanic-skin resistance sensor that measures electro-dermal resistance between two points. The computer 202 may also track key-logging information, movement information, click-rate information, and error rates related to use of the mouse 209. The keyboard may further include a wrist support that has a sensor embedded within it to measure conductivity to measure a user's heartrate and electrocardiogram (“EKG”) data. The computer 202 may also track key-logging information and error rates related to use of the keyboard.

In addition, the digital human workplace solution may be equipped with a photoplethysmogram to measure respirations. The photoplethysmogram may be included as part of pulse oximeter 210. The computer 202 is programmed to receive electronic data from the photoplethysmogram indicating the user's respiratory rate and from the pulse oximeter 210 indicating the user's oxygen saturation rate. The digital human displayed by computer 202 may be programmed to provide instructions to a user on how and when the use the photoplethysmogram, for example, the digital human may instruct the user to put their finger inside an attached pulse oximeter 210 and wait thirty seconds (or such other time is necessary to collect the measurements). While the user is performing those actions, the pulse oximeter 210 is measuring the saturation of oxygen in a person's blood and the included photoplethysmogram is measuring the respiratory rate of the user. The digital human may be programmed to respond to the user based on the oxygen saturation or respiratory rate of the user. For example, the digital human may receive information that the respiratory rate of the user is high or oxygen saturation low and accordingly instruct the user to take several deep breaths. In addition, the respiratory rate and oxygen saturation are recorded in an electronic health record accessible by other clinical staff to assist with further clinical evaluation.

The digital human workplace solution may also be equipped with floor scale 215, which measures a patient's weight. Floor scale 215 may be a pad using force sensor receptors embedded underneath carpet in the clinical setting such that it is not readily observable to the user. The digital human displayed by computer 202 may be programmed to provide instructions to a user on how and when the use the floor scale 215, for example, the digital human may instruct the user to stand still for the required amount of time, for example 10 seconds. Floor scale 215 may also take measurements to indicate whether the user is stable while communicating with the digital human.

The digital human workplace solution may also be equipped with payment collection equipment, such as a credit card reader or other point-of-sale hardware and software that enables the acceptance of payments.

The digital human workplace solution may also be equipped with other types of inputs to measure weather conditions, for example, a barometer to measure atmospheric pressure, a hygrometer for measuring humidity, or an anemometer for measuring wind speed.

Although in the context of FIG. 2, computer 202 is represented as a single computer, it is within the scope of the present invention for one or more computers to perform the functions discussed for computer 202. Further, the programming or software contemplated by the present invention is stored on a computer-readable medium.

Further, although FIG. 2 depicts the digital human workplace solution as a kiosk, other embodiments may use a computer application, phone app or virtual reality device, such as a virtual reality headset. In any of these embodiments, the computer, phone, or virtual reality device may be in electronic communication with one or more computers or input devices to perform the functions described herein.

The digital human workplace solution may be used in one embodiment to provide interactive training to individuals, such as required workplace training related to specific tasks.

In addition, although the digital human workplace solution is described as applicable to a clinical setting, it also may be applicable in other customer service settings. For example, the digital human workplace solution could be deployed at the border to screen immigrants. In such a situation, the ability of the digital human to switch languages based on the needs of the user is very important to obtaining useful information from the user.

The digital human workplace solution could also be deployed to answer frequently asked questions for students at a university, such as providing department-specific information, dorm information, schedule, routes between locations, and maps.

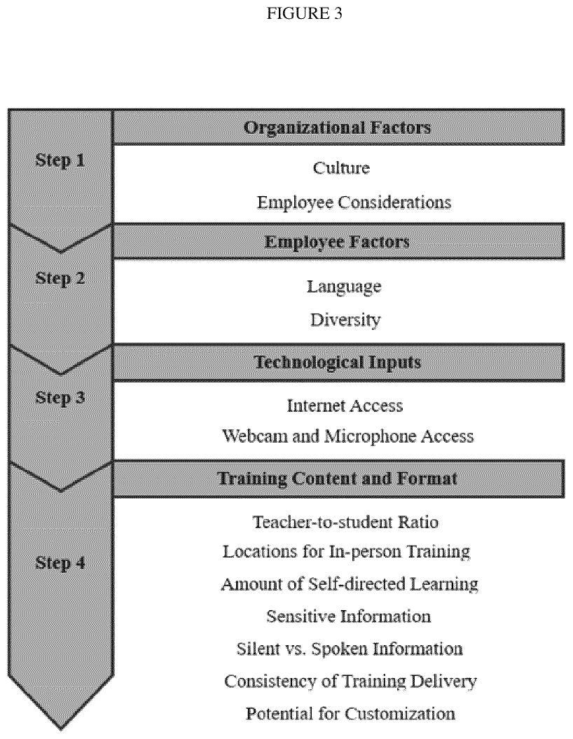

FIG. 3 depicts considerations for programming a digital human as a workplace trainer. For example, in Step 1, organizational factors may be considered such as the culture of the workplace and what roles the employees who are to be trained fulfill in that workplace.

As shown in Step 2 of FIG. 3, consideration may be given to what languages are spoken by the employees and employee diversity. For example, if the predominant languages spoken by employees are English and Spanish, the digital human may be programmed to ask the user whether they would prefer the training to occur in English or Spanish at the onset of the interaction. Providing training to employees in their native language promotes better understanding and can reduce costs associated with retraining. With respect to employee diversity, research suggests that individuals feel more at ease and have greater learning benefits when the instructor appears to share characteristics common to the individual's ethnicity, gender, or appearance.

In Step 3 of FIG. 3, the technological inputs available are considered, for example, what internet access is available to the digital human workplace solution (i.e., do employees all have access to strong, reliable Wi-Fi in all their work locations). While the digital human workplace solution may be programmed to support down to 2 megabits per second, it is preferred that a stronger internet connection is used to maximize the quality of the digital human interaction and prevent lags with the user in interacting with the training being provided by the digital human. Delays in the digital human understanding or responding to an employee can hinder the engagement, fluidity, and impact of the training.

In addition, the camera and microphone quality available to employees may be considered. While in one embodiment the digital human may provide audio output in response to text input, a preferred embodiment uses voice input for an immersive, engaged experience. Additionally, if a camera is turned on during the interaction, the digital human's programming can categorize the employee's facial features. This allows the digital human to alter features to mirror the employee, which can create feelings of rapport during the interaction. To take full advantage of this capability, access to a microphone and camera is required.

Finally, in Step 4 of FIG. 3, considerations regarding training content and format are taken into account when programming the subject matter for the training, including, but not limited to, the teacher-to-student ratio (i.e., will the digital human be interacting with one user at a time or multiple users at a time), locations for “in-person” training (i.e., where the digital human workplace solution will be available to the users for training—for example, there may be special considerations regarding privacy or reduction of ambient noise), the amount of self-directed learning (e.g., how much information will be given by the digital human before requiring feedback or a response from the user and how much flexibility will the user have to direct the order in which information is learned), sensitive information (e.g., does the outward expression of the digital human need to change when certain sensitive information is being conveyed), silent versus spoken information (i.e., should the digital human be programmed to pick up on both gestures and spoken word), the consistency of training delivery (i.e., should employees get all of the same information or is it acceptable for a subset of information to be provided to the user depending on the user's interest), and the potential for customization (i.e., as discussed more fully above, how much customization should the digital human be programmed with).

### Example 1

A digital human was programmed and deployed as follows. First, a fictional background and personal characteristics were established to assist in developing the personality of the digital human. In this example, the use case was specifically targeted at training office workers in the United States, who are typically in the 30-40+ age range. Therefore, a popular name from the 1990s, Cassandra, was chosen for the digital human. Cassandra's personality was developed using the HumanOS platform from Soul Machines, Inc. based on the words-revolutionary, extraordinary, innovative, scholastic, preventative, and professional.

Cassandra's dialog was written to reflect her personality as someone who cares about people, thinks the best of people, and wants them to improve/maintain their health. Her tone is upbeat, alert, and engaging. Cassandra is an empathetic, compassionate, and non-judgmental presence who is interested in people and looks forward to interacting with them.

An avatar shell was also chosen for Cassandra from stock shells available from Soul Machines. A female digital human was chosen that fit the warm, open personality that had been developed for Cassandra.

The original text of a traditional training provided to these office workers was transferred to a flowchart to organize conversation pathways and programming details for Google Dialogflow. The goal was not to have the digital human read every word of the traditional training but rather to create a conversation-based training in which the digital human and the user interact. To accomplish this goal, the spoken text of the digital human could not be too long. In order to prevent monotony, every 50-100 words spoken by the digital human had to be interspersed with questions asked toward the user. Additionally, not every conversation with the digital human could be the same; while the information covered would be the same, the digital human would be programmed to respond slightly different depending on how the user answered a question.

In Google Dialogflow, the digital human's dialogue, questions, and responses were created and logically linked together to form the different conversation pathways for each section in the training. Additionally, images, videos, menus with links, and website links were uploaded to be displayed on-screen alongside the digital human. In this example, original images and links from the traditional training were used. In addition, since digital humans are an emerging technology and many users may not immediately know how to interact with Cassandra, prompts were programmed to teach users when to speak with Cassandra and what to say.

The Google Dialogflow created was uploaded into the HumanOS Platform. Cassandra was additionally programmed to do real-time gesturing, have the “sweet” personality type, and respond with specific emotions to certain words and phrases (like “ergonomics” or “careful”). The “cinematic” camera view option was chosen, allowing the view being displayed to switch between close-up and gesturing shots.

Once Cassandra was ready to interact with users, usability tests were conducted with ten employees of the Texas A&M School of Public Health. Each employee underwent the training with Cassandra, which was monitored and analyzed with a usability testing process to note any programming issues. Feedback from the employees was also received after the completion of training. After each usability test, revisions were made to the Google Dialogflow programming until users could complete the training without any major glitches. This resulted in an average completion time for the digital human training program of 20-25 minutes, which was comparable to the average completion time for the traditional training.

The programmed training with Cassandra was then tested against the traditional training from which the dialog had been derived. Specifically, the pilot test involved ten users with half completing the traditional training and half completing the training with the digital human Cassandra. The users completed a study questionnaire before and after the trainings. Both the questionnaires and the trainings were found to be usable with no major errors.

### Example 2

In a second example, the programmed digital human is referred to as “Cassie” and interacts with the patient to assist with the check-in process for a medical visit. The system includes a display on which the digital human may be displayed, a microphone, a speaker, a camera or scanner, one or more databases to store patient information, and/or storage for versions of the programmed digital human personalized on a patient-by-patient basis. The system may also include one or more payment systems, including hardware required for such payment systems, such as a payment card reader. The system may further include additional input devices and related software such as a keyboard, cither a physical keyboard or a programmed digital keyboard, facial recognition software and/or hardware, and signature verification software.

In the first step, the programmed digital human introduces herself as follows: “Hi! My name is Cassie. I'll help you get checked into the appointment today. Would you please enter the patient's full name on the screen with given name first?” The patient may then input their name using a keyboard input, which may be either a physical or digital keyboard, or state their name out loud for recognition by the system. The system then may use the patient's name to determine whether this patient has previously interacted with the system. If so, a personalized version of Cassie may be accessed to further communicate with the patient. Based on a prior interaction, the personalized version of Cassie for a particular patient may use the patient's preferred language, communicate in a particular tone, and/or access information learned from the patient in one or more prior interactions to assist with the current interaction.

To confirm the patient's identity, Cassie responds in the next step “Thank you. Please tell me the patient's birthdate as month, day and year.” Following the patient's input of the birthdate either using a keyboard or by audibly stating their birthdate, the system will compare the birthdate to the name to verify that Cassie is accessing the correct medical record for the current patient. The system may also use facial recognition software to verify the identity of the patient.

Following identity confirmation, Cassie responds “Thank you. Now please scan the insurance card and drivers license.” The patient may then use an attached scanner to scan the insurance card and the driver's license. In an embodiment where Cassie is present on a mobile device, the mobile device's camera may be accessed in order to scan the insurance card and/or driver's license. The system may use the information collected in those scans to further verify the patient's identity and determine whether the patient has any balances due. In addition, information from the insurance card and the driver's license is collected and stored in a database, where it later may be accessed to, for example, assist with billing and collection efforts.

After confirming that the patient has scanned both the insurance card and the driver's license, Cassie responds, for example, “It looks like you have a balance of $50 on your account. Would you like to pay it in full right now?” Upon an affirmative response, Cassie says, “Okay, please insert your payment card” and waits for confirmation that the payment card has been inserted. The system may then use the payment card information to collect payment for the outstanding balance, and Cassie may, if necessary, ask the patient to provide their signature, either on the screen on which Cassie is being displayed or on a connected touchpad (connected to the system either wired or wireless).

In the next step, Cassie further interacts with the patient to verify the accuracy of their personal information, including phone number, address, preferred pharmacy information (such as phone number and address), and medical provider information. If the patient indicates that information needs to be updated, Cassie requests the updated information from the patient, and the system automatically updates that information in the patient's medical record.

Cassie may also interact with the patient to verify the reason for the current medical visit. For example, Cassie may ask the patient “Is today's appointment for an annual physical?” Based on the patient's answer, the system may then calculate the required co-pay due for the visit, by accessing information about co-pays relevant to the patient's insurance provider and plan from the database. Cassie may then relay the required co-pay to the patient, and a similar process to that described above for the outstanding balance is used to collect any co-pay due at the visit, either at the same time as the payment of the outstanding balance or separately.

Following the collection of all required patient identity and billing information, Cassie responds to the patient “Thank you! I have the patient checked in. Please take a seat in the lobby and the nurse will be right out.”

### Example 3

Beyond a personalized interaction to collect patient identity and billing information, the programmed digital human “Cassie” may also be used to collect medical data about the patient. In this example, the system is as noted in Example 2 and has additional inputs and programmed features. Cassie may interact with the patient to support the collection of medical data from the patient through a variety of input devices.

For example, the system is programmed to use the camera to track the patient's eye movements. Eye movement data may be used to alert the medical provider to inquire about the patient's stress or anxiety level during the visit if it is determined the patient is likely under stress. Specifically, the system may be programmed to detect eyelid twitching, which is an indicator of stress, and/or darting eyes, which may be an indicator of high anxiety. Cassie is programmed to inquire about the patient's stress or anxiety level if the eye movement data during the check-in process indicates eyelid twitching or darting eyes.

As another example, the system is connected to a thermometer such as a thermal scanning device, which collects the patient's temperature. Based on the determination of whether the patient has a fever (e.g., a temperature over a programmed threshold such as above 100.4 degrees Fahrenheit), Cassie instructs the patient to sit in a particular area of the waiting room (e.g., on the well patient side or sick patient side of the waiting room). In addition, the system automatically updates the patient's medical record to reflect the temperature taken on the day of the visit. If the temperature data indicates that the patient has a fever, Cassie is programmed to ask the patient questions about how long he or she has had a fever and what other symptoms the patient is experiencing, which then are used by the system to update the electronic medical record for that visit.

The system is also equipped with a floor scale and programmed to receive data from the floor scale and automatically record the weight of the user in an electronic medical record. The system compares the current patient weight to a prior patient weight recorded in the electronic medical record and records the change in weight in the electronic medical record, which is stored in a database and may be accessed by the medical provider during the visit. Cassie is programmed to inquire into reasons why the patient may have gained or lost weight.

The system is further equipped with a blood pressure monitor and programmed to receive data from the blood pressure monitor and automatically record the blood pressure of the user in an electronic medical record, which is stored in a database and may be accessed by the medical provider during the visit. The system compares the current blood pressure of the patient to the blood pressure of the patient taken previously to determine whether the patient's blood pressure has significantly changed and may store an alert for the medical provider in the electronic medical record if the patient's blood pressure has significantly changed.

The system is also equipped with a pulse oximeter, which is used to collect data about the patient's oxygen saturation rate, and a keyboard with a wrist support containing a sensor embedded within it to measure conductivity to determine the patient's heartrate and electrocardiogram (EKG) data. Cassie interacts with the patient to ensure that data can be collected from the pulse oximeter and conductivity sensor.

1. A digital human workplace solution system, comprising:
a. a computer containing program instructions on a computer-readable medium for displaying a digital human that is programmed to interact with a user;
b. a display; and
c. one or more devices to provide input data.

2. The digital human workplace solution system of claim 1, wherein at least one of the one or more devices to provide input data is connected to the computer via a wireless connection.

3. The digital human workplace solution system of claim 1, wherein at least one of the one or more devices to provide input data is a keyboard, a mouse, a pulse oximeter, a thermometer, a camera, a microphone, or a floor scale.

4. The digital human workplace solution system of claim 1, wherein at least one of the one or more devices to provide input data is a thermometer and the computer is further programmed such if the input data from the thermometer indicates the user has a fever, the digital human interacts with the user to seek additional input regarding additional symptoms or causes of the fever.

5. The digital human workplace solution system of claim 1, wherein at least one of the one or more devices to provide input data is a pulse oximeter and the computer is further programmed to receive data from the pulse oximeter and automatically record the oxygen saturation rate of the user in an electronic medical record.

6. The digital human workplace solution system of claim 1, wherein at least one of the one or more devices to provide input data is a floor scale and the computer is further programmed to receive data from the floor scale and automatically record the weight of the user in an electronic medical record.

7. The digital human workplace solution system of claim 1, wherein at least one of the one or more devices to provide input data is a camera, and the computer is programmed to receive data from the camera, process that data to determine at least one facial expression of the user, and automatically cause the displayed facial expression of the digital human to match the determined facial expression of the user.

8. The digital human workplace solution system of claim 1, wherein the display is a touchscreen.

9. The digital human workplace solution system of claim 1, wherein at least one of the one or more devices to provide input data is a mouse that is equipped with one or more of the following sensors: an accelerometer, a skin-temperature sensor, a pulse oximeter, or a galvanic-skin resistance sensor.

10. The digital human workplace solution system of claim 1, wherein at least one of the one or more devices to provide input data is a keyboard with a wrist support that includes a sensor to measure conductivity.

11. The digital human workplace solution system of claim 1, wherein at least one of the one or more device to provide input data is a photoplethysmogram and the computer is further programmed to receive data from the photoplethysmogram and automatically record the respiration rate of the user in an electronic medical record.

12. The digital human workplace solution system of claim 1, further comprising program instructions on a computer-readable medium for natural language processing.

13. The digital human workplace solution system of claim 1, further comprising program instructions on a computer-readable medium for converting speech to text.

14. The digital human workplace solution system of claim 1, further comprising program instructions on a computer-readable medium for converting text to speech.

15. The digital human workplace solution system of claim 1, further comprising one or more devices for payment collection.

16. A computer-implemented method comprising:
a. sending for display a digital human;
b. receiving data about a user from one or more input devices;
c. adjusting the dialog of the digital human in response to the received data.

17. The computer-implemented method of claim 16 further comprising causing at least one facial expression of the displayed digital human to match the facial expression of the user.

18. The method of claim 16, wherein the one or more devices is a keyboard, a mouse, a pulse oximeter, a thermometer, a camera, a microphone, or a floor scale.

19. A digital human workplace solution system, comprising:
a. one or more computers containing program instructions on a computer-readable medium for:
i. sending for display a digital human that is programmed to interact with a user;
ii. receiving data from one or more input devices connected to at least one of the one or more computers; and
iii. using the received data from at least one of the one or more input devices to automatically update the electronic medical record of the user;
b. a display;
c. a camera;
d. an infra-red thermometer;
e. a microphone;
f. a keyboard; and
g. a mouse.

20. The digital human workplace solution system of claim 19, further comprising one or more of the following: a scanner, payment collection equipment, a blood pressure monitor, a pulse oximeter, or a floor scale.
